SEQUENCE LISTING (36 sequences)

SEQ ID NO: 1 - DNA, 25 bp, Artificial Sequence
  primer ICP6gRNA4-F
caccggcctcggcgcagatcgatct

SEQ ID NO: 2 - DNA, 25 bp, Artificial Sequence
  primer ICP6gRNA4-R
aaacagatcgatctgcgccgaggcc

SEQ ID NO: 3 - DNA, 29 bp, Artificial Sequence
  primer MluI-gRNA-F
cgacgcgtgagggcctatttcccatgatt

SEQ ID NO: 4 - DNA, 44 bp, Artificial Sequence
  primer SpeI-sgRNA-R
gactagtcaataatcaatgtcacgggtacctctagagccatttg

SEQ ID NO: 5 - DNA, 474 bp, Artificial Sequence
  U6-ICP6gRNA fragment
cgacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcctcggcgcagatcgatctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgtgacattgattattgactagtc

SEQ ID NO: 6 - DNA, 22 bp, Artificial Sequence
  primer ICP6-SOE-F1
gcccaggctctggaccattacg

SEQ ID NO: 7 - DNA, 39 bp, Artificial Sequence
  primer ICP6-SOE-R1
gttatgtaacgcggaactcctcgatctgcgccgaggcgg

SEQ ID NO: 8 - DNA, 37 bp, Artificial Sequence
  primer ICP6-SOE-F3
ggcgtaaattgtaagcgtctcggacgtcagcgagggc

SEQ ID NO: 9 - DNA, 24 bp, Artificial Sequence
  primer ML40-SOE-R3
aaggttgttggtgcgaaggtaggc

SEQ ID NO: 10 - DNA, 35 bp, Artificial Sequence
  primer EGFP-SOE-F2
gcagatcgaggagttccgcgttacataacttacgg

SEQ ID NO: 11 - DNA, 40 bp, Artificial Sequence
  primer EGFP-SOE-R2
acgtccgagacgcttacaatttacgccttaagatacattg

SEQ ID NO: 12 - DNA, 1534 bp, Artificial Sequence
  upstream homology arm
gcccaggctctggaccattacgactgtctgatccacagcacaccgcacacgctggtcgagcgggggctgcaatcggccctgaagtatgaggagttttacctaaagcgctttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccatcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaatctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatcgaggagttccgcgttacataac

SEQ ID NO: 13 - DNA, 1194 bp, Artificial Sequence
  downstream homology arm
ggcgtaaattgtaagcgtctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaaccccctccgcgccaggcccgccgccactgtcgtcgccgtcccacgcgctcccccgctgccatggattccgcggccccagccctctcccccgctctgacggcccatacgggccagagcgcgccggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgaccaggcgcgccgcgagtacgtggccggcaccatcaaccacccggccatccgcgccaaggtggactggctggaagcgcgggtgcgggaatgtgcctccgttccggaaaagttcatcctcatgatcctcatcgagggcatcttttttgccgcctcgtttgccgccatcgcctaccttcgcaccaacaacctt

SEQ ID NO: 14 - DNA, 1628 bp, Artificial Sequence
  foreign EGFP gene
gcagatcgaggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgtctcggacgt

SEQ ID NO: 15 - DNA, 4300 bp, Artificial Sequence
  Targeting gene
gcccaggctctggaccattacgactgtctgatccacagcacaccgcacacgctggtcgagcgggggctgcaatcggccctgaagtatgaggagttttacctaaagcgctttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccatcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaatctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatcgaggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgtctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcacgagctgcgcgctgtgaccgacaaaccccctccgcgccaggcccgccgccactgtcgtcgccgtcccacgcgctcccccgctgccatggattccgcggccccagccctctcccccgctctgacggcccatacgggccagagcgcgccggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgctttttcacaacaacgaccaggcgcgccgcgagtacgtggccggcaccatcaaccacccggccatccgcgccaaggtggactggctggaagcgcgggtgcgggaatgtgcctccgttccggaaaagttcatcctcatgatcctcatcgagggcatcttttttgccgcctcgtttgccgccatcgcctaccttcgcaccaacaacctt

SEQ ID NO: 16 - DNA, 27 bp, Artificial Sequence
  primer U6-seq-F2
tgcatatacgatacaaggctgttagag

SEQ ID NO: 17 - DNA, 71 bp, Artificial Sequence
  HSV-1 partial ICP6 gene containing a GATC insertion
gccagtttgtcgcgctgatgcccaccgccgcctcggcgcagatcgatctcggacgtcagcgagggctttgc

SEQ ID NO: 18 - DNA, 20 bp, Artificial Sequence
  ICP6 target sequence 1 for gRNA recognition
atcgatctgcgccgaggcgg

SEQ ID NO: 19 - DNA, 20 bp, Artificial Sequence
  ICP6 target sequence 2 for gRNA recognition
gagatcgatctgcgccgagg

SEQ ID NO: 20 - DNA, 20 bp, Artificial Sequence
  ICP6 target sequence 3 for gRNA recognition
tccgagatcgatctgcgccg

SEQ ID NO: 21 - DNA, 20 bp, Artificial Sequence
  ICP6 target sequence 4 for gRNA recognition
gcctcggcgcagatcgatct

SEQ ID NO: 22 - DNA, 20 bp, Artificial Sequence
  ICP6 target sequence 5 for gRNA recognition
atcgatctcggacgtcagcg

SEQ ID NO: 23 - DNA, 20 bp, Artificial Sequence
  ICP6 target sequence 6 for gRNA recognition
tcgatctcggacgtcagcga

SEQ ID NO: 24 - RNA, 20 nt, Artificial Sequence
  guide sequence of gRNA 1
uagcuagacgcggcuccgcc

SEQ ID NO: 25 - RNA, 20 nt, Artificial Sequence
  guide sequence of gRNA 2
cucuagcuagacgcggcucc

SEQ ID NO: 26 - RNA, 20 nt, Artificial Sequence
  guide sequence of gRNA 3
aggcucuagcuagacgcggc

SEQ ID NO: 27 - RNA, 20 nt, Artificial Sequence
  guide sequence of gRNA 4
cggagccgcgucuagcuaga

SEQ ID NO: 28 - RNA, 20 nt, Artificial Sequence
  guide sequence of gRNA 5
uagcuagagccugcagucgc

SEQ ID NO: 29 - RNA, 20 nt, Artificial Sequence
  guide sequence of gRNA 6
agcuagagccugcagucgcu

SEQ ID NO: 30 - DNA, 21 bp, Artificial Sequence
  primer ICP6-F
tgtcggcgatgaagaccagca

SEQ ID NO: 31 - DNA, 19 bp, Artificial Sequence
  primer ICP6-R
gcggaccagggtggaggct

SEQ ID NO: 32 - DNA, 22 bp, Artificial Sequence
  primer HSV1-GFP-KI-F1
gaccactaccagcagaacaccc

SEQ ID NO: 33 - DNA, 20 bp, Artificial Sequence
  primer HSV1-GFP-KI-R1
cgaacaaacgacccaccaat

SEQ ID NO: 34 - DNA, 19 bp, Artificial Sequence
  primer HSV1-GFP-KI-F2
gcccgacaaccactacctg

SEQ ID NO: 35 - DNA, 22 bp, Artificial Sequence
  primer HSV1-GFP-KI-R2
cgtccctgacaagaatcacaat

SEQ ID NO: 36 - DNA, 10076 bp, Artificial Sequence
  pCas9-ICP6gRNA-NeoR
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcctcggcgcagatcgatctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgccaccatggccccaaagaagaagcggaaggtcggtatccacggtgtcccagcagccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatactcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagcaggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaagcgtcctgctgctactaagaaagctggtcaagctaagaaaaagaaataatctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

(19) United States
(12) Patent Application Publication
(10) Pub. No.: US 2022/0017897 A1
(43) Pub. Date: Jan. 20, 2022

(54) GUIDE RNA FOR HSV-1 GENE EDITING AND METHOD THEREOF

(71) Applicant: Oriengene Biotechnology Ltd, Beijing (JP)

(72) Inventors: Xiaoming Li, Beijing (CN); Xiao Shu, Beijing (CN); Ru Jia, Beijing (CN); Ning Wang, Beijing (CN)

(21) Appl. No.: 17/040,796

(22) PCT Filed: Jul. 20, 2020
(86) PCT No.: PCT/CN2020/100684

§ 371 Date: Sep. 23, 2020

(51) Int. Cl. C12N 15/113; C12N 9/22

(52) U.S. Cl. CPC C12N 15/113; C12N 2310/20; C12N 9/22

(57) ABSTRACT

The invention relates to a guide RNA (gRNA) comprising a guide sequence capable of targeting a Cas9 protein to a target sequence in an ICP6 gene of type 1-herpes simplex virus (HSV-1) to provide a specific cleavage event, wherein the target sequence comprises a GATC insertion as compared to a wild-type HSV-1. The invention also relates to a HSV-1 gene-editing system and a gene-editing method for generating a desired recombinant HSV-1 by using said gRNA.

### FIELD

The herein disclosed embodiments relate to the field of gene editing, especially to an artificial guide RNA (gRNA) for Cas9-mediated gene editing of type-1 herpes simplex virus (HSV-1). Further disclosed is a Cas9-mediated gene-editing method by using said gRNA.

### BACKGROUND

121 Type-1 herpes simplex virus (HSV-1; also known by their taxonomical names Human alphaherpesvirus 1) is a double-stranded DNA virus in the genus Simplexvirus, and family Herpesviridae. HSV-1 contains 152,000 nucleotides. In the past decades. HSV-1 has been modified to generate various oncolytic viruses for treating cancers. Among them, the recombinant HSV-1 strain. OrienX010 (ICP34.5−/−, ICP47−, ICP6−), is an oncolytic virus under an ongoing clinical trial, which comprises a human GM-CSF gene inserted in two copies at the original ICP34.5 gene and an unpublished deactivation modification within ICP6 gene.

CRISPR/Cas9-mediated homologous recombination is used to improve gene editing of HSV-1, especially for gene insertion (Dong Wang et al. (2018), Cancer Gene Ther, 25(5-6):93-105). However, due to differences among various HSV-1 mutants, existing gene-editing methods are not capable of being applied to all kinds of HSV-1 mutant. In particular, using a CRISPR/Cas9 system to edit the modified ICP6 gene region of said HSV-1 mutant OrienX010 cannot be readily achieved.

At present there is still a need for developing an efficient gene-editing method for such kind of HSV-1 mutant.

### SUMMARY

The herein disclosed embodiments relate to a guide RNA (gRNA) comprising a guide sequence capable of targeting a Cas9 protein to a target sequence in an ICP6 gene of type 1-herpes simplex virus (HSV-1) to provide a specific cleavage event, wherein the target sequence comprises a GATC insertion as compared to a wild-type HSV-1.

Some embodiments relate to a first polynucleotide encoding a gRNA as described above.

Some embodiments also relate to a vector comprising a first polynucleotide, as described above, operably linked to a suitable promoter, and optionally further comprises a second polynucleotide encoding a Cas9 protein.

Some embodiments also relate to a vector system comprising one vector as described above and a second vector comprising a second polynucleotide encoding a Cas9 protein.

Some embodiments also relate to a transformant cell transformed with a vector or a vector system as described above, which is capable of expressing a gRNA and a Cas9 protein.

Some embodiments also relate to a Cas9/gRNA complex comprising a gRNA as described above and a Cas9 protein.

Some embodiments also relate to a gene-editing system for HSV-1, which comprises:
(a) a HSV-1 strain comprising a target sequence in an ICP6 gene, wherein the target sequence comprises a GATC insertion as compared to a wild-type HSV-1;
(b) a vector or a vector system as described above, which is capable of expressing a gRNA and a Cas9 protein; and
(c) a targeting polynucleotide comprising an upstream homology arm, an exogenous gene and a downstream homology arm sequentially, wherein the upstream homology arm and the downstream homology arm are separately homologous to a 5′ region and a 3′ region of the target domain of said HSV-1, wherein the target sequence is located within the target domain of said HSV-1.

The present invention also relates to a gene-editing method for generating a recombinant HSV-1, which comprises steps of:
(a) providing a HSV-1 strain, wherein the HSV-1 strain comprises a target sequence in an ICP6 gene, and the target sequence comprises a GATC insertion as compared to a wild-type HSV-1;
(b) constructing a vector or a vector system as described above, which is capable of expressing a gRNA and a Cas9 protein;
(c) preparing a linear DNA including a targeting polynucleotide, which is capable of inserting an exogenous gene into the target domain of said HSV-1 via homologous recombination;
(d) transforming the vector or the vector system into a cell to obtain a first transformant cell;
(e) transforming the linear DNA into the first transformant cell to obtain a second transformant cell;
(f) infecting the second transformant cell with the HSV-1 strain to cause CRISPR/Cas9-mediated homologous recombination to occur in the second transformant cell, wherein the gRNA targets the Cas9 protein to the target sequence to provide a specific cleavage event, and then the exogenous gene is inserted into the target domain via homologous recombination.

The present invention also relates to a recombinant HSV-1 generated by the gene-editing method as described above.

### BRIEF DESCRIPTION OF THE DRAWINGS

FIG. 1 shows a sequence alignment comparison of HSV-1 target strain's partial ICP6 sequence (SEQ ID No. 17) and HSV-1 strain 17.

FIG. 2 shows a gel electrophoresis result of gRNA cleavage efficiency assay. Lane 1 contains a DNA marker, lane 2 to 7 shows the results for gRNA 1 to 6, and lane 8 contains a positive control provided in Cas9 in-vitro cleavage kit (Inovogen Tech. Co., catalog No. PC1400).

FIG. 3 shows a gene map of plasmid “pCas9-ICP6gRNA-Neo^{R}”.

FIG. 4 is a diagram showing the concept of Cas9/gRNA-mediated homologous recombination of the invention to insert EGFP into the ICP6 locus in HSV-1.

FIG. 5 shows a gel electrophoresis result of two stage PCR identification of obtained recombinant HSV-1. Lanes 2-6 are results of first stage PCR (with primer pairs HSV1-GFP-KI-F1 and HSV1-GFP-KI-R1), wherein lane 2-5 contain the PCR products from recombinant HSV-1 samples 1-4 respectively, and lane 6 contains the PCR products from the HSV-1 target strain. Lanes 8-12 are results of second stage PCR (with primer pairs HSV1-GFP-KI-F2 and HSV1-GFP-KI-R2), wherein lane 8-11 contain the PCR products from recombinant HSV-1 samples 1-4 respectively, and lane 12 contains the PCR products from the HSV-1 target strain. Lanes 1, 7 and 13 contain DNA markers.

FIG. 6 shows a result of plaque purification. Green fluorescent plaques are indicated by gray arrows, and plaques without green fluorescence are indicated by black arrows. The upper panel showed the result observed by fluorescence microscope; the lower panel showed the result observed by non-fluorescence optical microscope.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

### Definition

As used herein, the term “guide RNA”, “gRNA”, “single guide RNA” and “sgRNA” are used interchangeably herein and referr to an RNA molecule (or a group of RNA molecules collectively) that can bind to a Cas protein, and aid in targeting the Cas protein to a specific location within a target sequence (e.g., a DNA sequence). A non-naturally occurring (artificial) gRNA that has “gRNA functionality” is one that has one or more of the functions of naturally occurring guide RNA, such as associating with a Cas protein, or a function performed by the guide RNA in association with a Cas protein to provide a specific cleavage event.

The term “CRISPR-associated protein” or “Cas protein” refers to a wild type Cas protein, a fragment thereof, or a variant thereof. The term “Cas9” or “Cas9 protein” or “Cas9 nuclease” or “CRISPR-associated protein 9” refers to an RNA-guided DNA nuclease, a fragment thereof, or a mutant or variant thereof, which is capable of associating with a gRNA and cleave a target DNA sequence. Generally, a Cas9 protein comprises a gRNA binding domain and a DNA cleavage domain.

In some embodiments, Cas9 protein is a RNA-guided DNA nuclease which recognizes a PAM sequence (i.e. NGG). In some embodiments, Cas9 protein is a RNA-guided DNA nuclease derived from Streptococcus pyogenes.

The term “Cas9/gRNA complex” refers to a complex containing a Cas9 protein and a guide RNA, which could cooperatively provide a specific cleavage event at a target DNA.

The term “guide sequence” refers to a sequence of guide RNA complementary to a target sequence of a target DNA. Generally, a guide sequence is a RNA molecule about 20 nucleotides consisted of A, U, C and G.

The term “target sequence” refers to a DNA sequence to be targeted and cleaved by a Cas9/gRNA complex. The target sequence is complementary to a guide sequence of gRNA, and located at 5′ end of a protospacer-adjacent motif (PAM) sequence. The target sequence is a sequence about 15-25 nucleotides. In some embodiments, the target sequence is a sequence of 20 nucleotides.

The term “protospacer-adjacent motif (PAM)” refers to a 2-6 base pair DNA sequence immediately following the target sequence targeted by the Cas9 protein and help the Cas9 protein to provide a specific cleavage event. For example, PAM sequence is NGG, such as CGG, AGG, TGG and GGG.

The term “specific cleavage event” refers to a specific DNA cleavage caused by a Cas9/gRNA complex, and there is no off-target effect occurred during the cleavage process.

The term “vector” refers to a nucleic acid molecule capable of transporting another nucleic acid to which it has been linked. One type of vector is a “plasmid,” which refers to a circular double stranded DNA loop into which additional DNA segments can be inserted, such as by standard molecular cloning techniques.

The term “transformant cell” refers to a cell which has been transformed or transfected with an extracellular DNA (exogenous, artificial or modified), and could expresses the gene(s) contained therein.

The term “homology directed repair (HDR)” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “homologous recombination (HR)” refers to the crossing over of DNA that occurs between two homologous DNA molecules. Homologous recombination is a common method for inserting an exogenous gene or deleting a desired gene in a cell.

The term “targeting polynucleotide” refers to a synthesized sequence used for crossing over of DNA via homologous recombination, which generally comprises an upstream homology arm, a downstream homology arm, and optionally an exogenous gene (depending on purpose of insertion or deletion). The term “targeting vector” refers to a vector comprising a targeting polynucleotide.

The term “target domain” refers to a sequence of HSV-1 which is selected for homologous recombination. The target domain usually comprises a 5′ region homologous to an upstream homology arm of a targeting polynucleotide, and a 3′ region homologous to a downstream homology arm of the targeting polynucleotide. The target sequence is located within the target domain of HSV-1, wherein a specific cleavage event occurred in the target sequence would enhance the homologous recombination in the target domain.

### DETAILED DESCRIPTION

In detail, the present invention relates to an artificial guide RNA (gRNA) comprising a guide sequence capable of targeting a Cas protein to a target sequence in an ICP6 gene of type 1-herpes simplex virus (HSV-1) to provide a specific cleavage event, wherein the target sequence comprises a GATC insertion as compared to a wild-type HSV-1. Particularly, the Cas protein used herein is a Cas9 protein.

In one aspect, the present invention relates to a gRNA as described above, wherein the ICP6 gene expresses an inactivated ICP6 protein caused by inserting a GATC sequence. In another aspect, the ICP6 gene is an inactivated ICP6 gene.

In one embodiment, the ICP6 gene comprises a sequence corresponding to nucleotides 25-64 of SEQ ID NO. 17. In another embodiment, the ICP6 gene comprises SEQ ID NO. 17.

The HSV-1 applied herein could be any HSV-1 mutant which has an ICP6 gene feature as described above, i.e. GATC insertion. In one embodiment, the HSV-1 applied herein comprises a sequence corresponding to nucleotides 25-64 of SEQ ID NO. 17. In one embodiment, the HSV-1 applied herein comprises SEQ ID NO. 17. For example, the HSV-1 used could be a wild type HSV-1 or HSV-1 stain CL-1 (CGMCC 1736) or HSV-1 strain 17, which is inserted an GTAC sequence into its ICP6 gene to result in SEQ ID NO. 17.

In another aspect, the HSV-1 used could be any existing modified HSV-1 in which is inserted an GTAC sequence in its ICP6 gene to result in SEQ ID NO. 17. For example, the HSV-1 used could be strain OrienX010, which comprises a GATC sequence inserted into its ICP6 gene to result in SEQ ID NO. 17.

In another aspect, the present invention refers to a gRNA as described above, wherein the target sequence is a sequence of a 20 nucleotides segment of nucleotides 25-64 of SEQ ID NO. 17. In one embodiment, the target sequence comprises a sequence of a 20 nucleotides segment of SEQ ID NO. 17.

In another embodiment, the target sequence comprises a sequence selected from the group consisting of SEQ ID No. 18, 19, 20, 21, 22 and 23 In a particular embodiment, the target sequence is selected from the group consisting of SEQ ID No. 18, 19, 20, 21, 22 and 23 For example, the target sequence is SEQ ID No. 21.

In another aspect, the present invention refers to a gRNA as described above, wherein the guide sequence comprises a sequence selected from the group consisting of SEQ ID No. 24, 25, 26, 27, 28 and 29. In one embodiment, the guide sequence is a sequence selected from the group consisting of SEQ ID No. 24, 25, 26, 27, 28 and 29. For example, the guide sequence is SEQ ID No. 27.

Generally, an artificial gRNA comprises a guide sequence which is complementary to a target sequence in a target gene, and a Cas9-associated sequence which binds to a Cas9 protein and forms a Cas9/gRNA complex for causing target sequence cleavage. In some embodiments, the guide sequence is complementary to a target sequence in ICP6 gene of HSV-1. A DNA sequence encoding a Cas9-associated sequence of a gRNA is, for example, gttttagagctagaaatagcaagttaaaataaggctagtccgttatcaattgaaagtggcaccgagtcggtgctttt.

In a particular aspect, the present invention relates to a first polynucleotide encoding a gRNA, wherein the gRNA comprises a guide sequence and a Cas9-associated sequence as described above.

In a particular aspect, the present invention relates to a vector comprising a first polynucleotide encoding a gRNA as described above operably linked to a suitable promoter, and optionally further comprises a second polynucleotide encoding a Cas9 protein.

Particularly, the present invention relates to a vector comprising a first polynucleotide encoding a gRNA as described above operably linked to a suitable promoter, and a second polynucleotide encoding a Cas9 protein. For example, the vector of the invention could be a plasmid pCas9-ICP6gRNA-Neo^{R} as constructed in example 3. In another aspect, the suitable promoter for expressing gRNA could be an U6 promoter.

In a particular aspect, the present invention relates to a vector system comprising a first vector and a second vector, wherein the first vector comprises a first polynucleotide encoding a gRNA as described above, and the second vector comprises a second polynucleotide encoding a Cas9 protein.

In another aspect, the present invention relates to a transformant cell transformed with a vector or a vector system as described above, which is capable of expressing a gRNA and a Cas9 protein. In particular, the transformant cell used herein includes, but not limited to, a Vero cell, a BHK cell or a HEK293 cell. In one embodiment, the transformant cell is a Vero cell.

In a particular aspect, the present invention relates to a Cas9/gRNA complex comprising a gRNA as described above and a Cas9 protein.

In a particular aspect, the present invention relates to a gene-editing system for HSV-1, which comprises:
(a) a HSV-1 strain comprising a target sequence in an ICP6 gene, wherein the target sequence comprises a GATC insertion as compared to a wild-type HSV-1;
(b) a vector or a vector system as described above, which is capable of expressing a gRNA as described above and a Cas9 protein; and
(c) a targeting polynucleotide comprising an upstream homology arm, an exogenous gene and a downstream homology arm sequentially, wherein the upstream homology arm and the downstream homology arm are separately homologous to 5′ region and 3′ region of a target domain of said HSV-1, wherein the target sequence is located within the target domain of said HSV-1.

Ina particular aspect, the present invention relates to a gene-editing method for generating a recombinant HSV-1, which comprises steps of:
(a) providing a HSV-1 strain, wherein the HSV-1 strain comprises a target sequence in an ICP6 gene, and the target sequence comprises a GATC insertion as compared to a wild-type HSV-1;
(b) constructing a vector or a vector system as described above, which is capable of expressing a gRNA as described above and a Cas9 protein;
(c) preparing a linear DNA including a targeting polynucleotide, which is capable of inserting an exogenous gene into a target domain of said HSV-1 via homologous recombination;
(d) transforming the vector or the vector system into a transformant cell to obtain a first transformant;
(e) transforming the linear DNA into the first transformant to obtain a second transformant;
(f) infecting the second transformant with the HSV-1 strain to cause CRISPR/Cas9-mediated homologous recombination occurred in the transformant, wherein the gRNA targets the Cas9 protein to the target sequence to provide a specific cleavage event, and then the exogenous gene is inserted into the target domain via homologous recombination.

In the gene-editing method of the invention, the ICP6 gene is an inactivated ICP6 gene caused by inserting a GATC sequence. In another embodiment, the ICP6 gene is partially deleted. In a particular embodiment, the ICP6 gene comprises a sequence corresponding to nucleotides 25-64 of SEQ ID NO. 17. Particularly, the ICP6 gene comprises SEQ ID NO. 17.

In the gene-editing method of the invention, the target sequence comprises a sequence selected from the group consisting of SEQ ID No. 18, 19, 20, 21, 22 and 23. In one embodiment, the target sequence is selected from the group consisting of SEQ ID No. 18, 19, 20, 21, 22 and 23. In a particular embodiment, the target sequence is SEQ ID No. 21.

In particular, the linear DNA in step (c) is obtained by digesting a homologous recombination (HR) targeting vector including the targeting polynucleotide with a restriction enzyme. The HR targeting vector used for inserting a desired targeting polynucleotide could be a commercial vector. e.g. pEASY-Blunt cloning vector.

In particular, the targeting polynucleotide in step (c) comprises an upstream homology arm, the exogenous gene and a downstream homology arm sequentially; wherein the upstream homology arm and the downstream homology arm are separately homologous to a 5′ region and a 3′ region of a target domain of said HSV-1, wherein the target sequence is located within the target domain of said HSV-1.

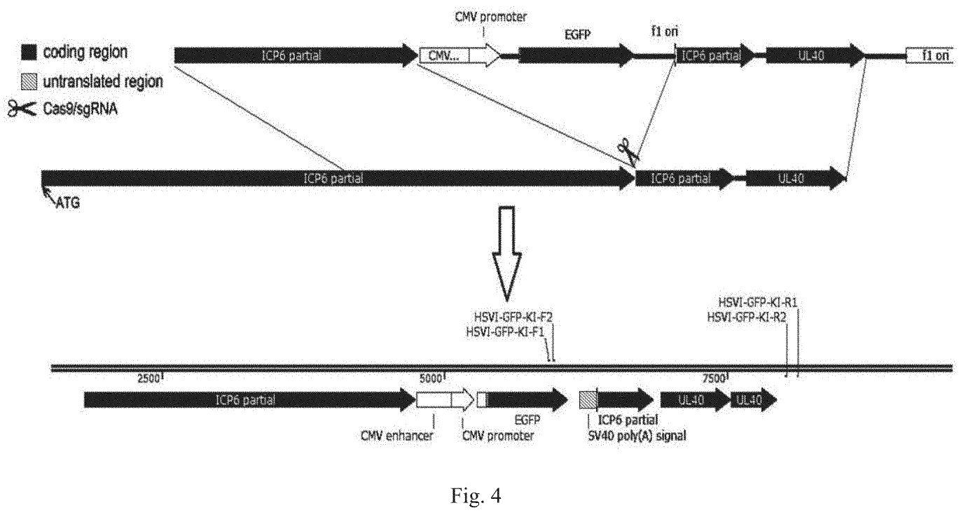

Particularly, the target domain of said HSV-1 comprises an upstream homology arm at 5′ region and a downstream homology arm at 3′ region, wherein the target sequence being cleaved by Cas9/gRNA system is located within the target domain. In one embodiment, the target domain of said HSV-1 comprises a partial ICP6 gene segment and an UL40 gene, wherein the upstream homology arm is homologous to an upstream sequence of the partial ICP6 gene segment, the downstream homology arm is homologous to a downstream sequence of the partial ICP6 gene segment and the UL40 gene, and the target sequence being cleaved by Cas9/gRNA system is located within the partial ICP6 gene segment (as shown in FIG. 4).

The upstream homology arm is homologous to a 5′ region of the target domain of said HSV-1. In one embodiment, the upstream homology arm comprises a sequence of SEQ ID No. 12. The downstream homology arm is homologous to a 3′ region of the target domain of said HSV-1. In one embodiment, the downstream homology arm comprises a sequence of SEQ ID No. 13.

The exogenous gene used herein includes, but not limited to, a gene which can encode (1) a reporter, such as green fluorescent protein (GFP), enhanced green fluorescent protein (EGFP), mCherry, DsRed, tdTomato or ZsGreen; (2) an immune regulatory cytokine, such as GM-CSF, interleukins (i.e., IL-1, IL-2, IL-3, IL-4, IL-5, IL-6, IL-7, IL-8, IL-9 and IL-10), interferons (IFN) and tumor necrosis factor (i.e., TNF-α and TNF-β); (3) a therapeutic antibody or a functional fragment thereof, such as anti-PD-1 antibody, anti-PD-L1 antibody, anti-CTLA4 antibody, anti-LAG3 antibody and anti-TIM3 antibody; (4) a fusion protein for modulating an immune response; (5) a chemokine, such as CCL5, CCL20 and CCL21; (6) a tumor apoptotic-related factor, such as tumor necrosis factor-(TNF)-related apoptosis-inducing ligand (TRAIL) and P53 gene; (7) an anti-angiogenesis factor, such as endostatin and vascular endothelial growth inhibitor (VEGI); (8) a small RNA for inhibiting tumor-related gene expression, such as miRNA, siRNA, shRNA and lncRNA; or (9) a tumor-associated antigen (TAA) or tumor-specific antigen (TSA), such as alphafetoprotein (AFP), melanoma-associated antigen (MAGE), HER2, EGFR, PSA, TRP-2, EpCAM, GPC3, mesothelin (MSLN), CD20, CD40, and PD-L1.

In a particular aspect, the present invention relates to a recombinant HSV-1 generated by the gene-editing method as described above.

### EXAMPLES

The present invention is further exemplified, but not limited, by the following examples that illustrate the gRNA and HSV-1 gene-editing method of the invention.

### Materials

HSV-1 strain CL-1 was deposited in China General Microbiological Culture Collection Center (CGMCC) with a deposition number of CGMCC 1736 on Jun. 14, 2006, which has a 99.5% sequence similarity with HSV-1 stain 17 (NCBI accession number: NC_001806).

The genome of HSV-1 strain CL-1 (151180 bp) was modified by using a homologous recombination method as described in CN1283803C or CN101376893B to obtain HSV-1 strain OrienX010 (ICP34.5^{-/-}, ICP47⁻, ICP6⁻) (hereinafter referred to as “HSV-1 target strain”). In brief, such genetic modification included deletions of ICP34.5 genes (two copies at 513-1259 nucleotides and 123999-124745 nucleotides) and ICP47 gene (at 144230-144496 nucleotides), as well as an insertion of a human GM-CSF gene into the original position of ICP34.5 genes. In addition, a GATC sequence was inserted at the site between 88251-88252 nucleotides (wherein original ICP6 gene was located at 85327-88740 nucleotides) to result in a frameshift mutation as well as inactivation of said ICP6 gene. Therefore, the obtained HSV-1 target strain has a partial ICP6 sequence as shown in SEQ ID No. 17, wherein SEQ ID No. 17 contains said GATC insertion.

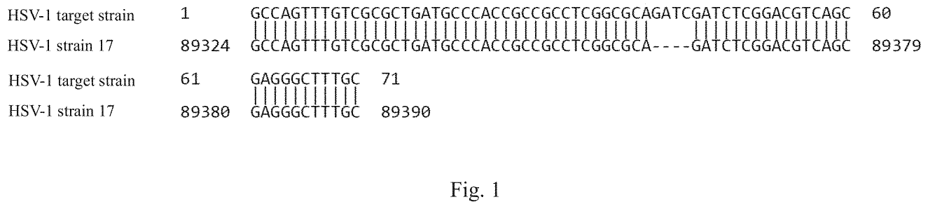

As shown in FIG. 1, a sequence alignment comparison of HSV-1 target strain's partial ICP6 sequence (SEQ ID No. 17) and HSV-1 strain 17 (NCBI accession number: NC_001806) was conducted. The partial ICP6 sequence (SEQ ID No. 17) of HSV-1 target strain is identical to 89324-89390 nucleotides of HSV-1 strain 17's genome DNA, except for the GATC insertion. The obtained HSV-1 target strain and its genome DNA were purified and stored in a suitable condition for the following experiments.

Sequences used herein are summarized in Table 1 for quick review.

TABLE 1

| SEQ | Component/ | Length |
|---|---|---|
| ID No. | sequence (5′→3′) | (nt) |
| 1 | ICP6gRNA4-F | 25 |
| | CACC GGCCTCGGCGCAGATCGATCT | |
| 2 | ICP6gRNA4-R | 25 |
| | AAAC AGATCGATCTGCGCCGAGGCC | |
| 3 | MluI-gRNA-F | 29 |
| | CGACGCGTGAGGGCCTATTTCCCATGATT | |
| 4 | SpeI-sgRNA-R | 44 |
| | GACTAGTCAATAATCAATGTCACGGGTACCTC | |
| | TAGAGCCATTTG | |
| 5 | U6-ICP6gRNA fragment | 474 |
| 6 | ICP6-SOE-F1 | 22 |
| | GCCCAGGCTCTGGACCATTACG | |
| 7 | ICP6-SOE-R1 | 39 |
| | GTTATGTAACGCGGAACTCCTCGATCTGCGCC | |
| | GAGGCGG | |
| 8 | ICP6-SOE-F3 | 37 |
| | GGCGTAAATTGTAAGCGTCTCGGACGTCAGCG | |
| | AGGGC | |
| 9 | ML40-SOE-R3 | 24 |
| | AAGGTTGTTGGTGCGAAGGTAGGC | |
| 10 | EGFP-SOE-F2 | 35 |
| | GCAGATCGAGGAGTTCCGCGTTACATAACTTA | |
| | CGG | |
| 11 | EGFP-SOE-R2 | 40 |
| | ACGTCCGAGACGCTTACAATTTACGCCTTAAG | |
| | ATACATTG | |
| 12 | upstream homology arm (UHA) | 1534 |
| 13 | downstream homology arm (DHA) | 1194 |
| 14 | exogenous EGFP gene | 1628 |
| 15 | Targeting polynucleotide | 4300 |
| | (containing UHA, exogenous EGFP | |
| | gene, and DHA) | |
| 16 | U6-seq-F2 | 27 |
| | TGCATATACGATACAAGGCTGTTAGAG | |
| 17 | HSV-1 partial ICP6 sequence | 71 |
| | containing a GATC insertion | |
| | GCCAGTTTGTCGCGCTGATGCCCACCGCCGCC | |
| | TCGGCGCAGATCGATCTCGGACGTCAGCGAGG | |
| | GCTTTGC | |
| 18 | ICP6 target sequence 1 for gRNA | 20 |
| | recognition | |
| | ATCGATCTGCGCCGAGGCGG | |
| 19 | ICP6 target sequence 2 for gRNA | 20 |
| | recognition | |
| | GAGATCGATCTGCGCCGAGG | |
| 20 | ICP6 target sequence 3 for gRNA | 20 |
| | recognition | |
| | TCCGAGATCGATCTGCGCCG | |
| 21 | ICP6 target sequence 4 for gRNA | 20 |
| | recognition | |
| | GCCTCGGCGCAGATCGATCT | |
| 22 | ICP6 target sequence 5 for gRNA | 20 |
| | recognition | |
| | ATCGATCTCGGACGTCAGCG | |
| 23 | ICP6 target sequence 6 for gRNA | 20 |
| | recognition | |
| | TCGATCTCGGACGTCAGCGA | |
| 24 | guide sequence of gRNA 1 | 20 |
| | UAGCUAGACGCGGCUCCGCC | |
| 25 | guide sequence of gRNA 2 | 20 |
| | CUCUAGCUAGACGCGGCUCC | |
| 26 | guide sequence of gRNA 3 | 20 |
| | AGGCUCUAGCUAGACGCGGC | |
| 27 | guide sequence of gRNA 4 | 70 |
| | CGGAGCCGCGUCUAGCUAGA | |
| 28 | guide sequence of gRNA 5 | 20 |
| | UAGCUAGAGCCUGCAGUCGC | |
| 29 | guide sequence of gRNA 6 | 20 |
| | AGCUAGAGCCUGCAGUCGCU | |
| 30 | ICP6-F | 21 |
| | TGTCGGCGATGAAGACCAGCA | |
| 31 | ICP6-R | 19 |
| | GCGGACCAGGGTGGAGGCT | |
| 32 | HSV1-GFP-K1-F1 | 22 |
| | GACCACTACCAGCAGAACACCC | |
| 33 | HSV1-GFP-K1-R1 | 20 |
| | CGAACAAACGACCCACCAAT | |
| 34 | HSV1-GFP-K1-F2 | 19 |
| | GCCCGACAACCACTACCTG | |
| 35 | HSV1-GFP-K1-R2 | 22 |
| | CGTCCCTGACAAGAATCACAAT | |
| 36 | pCas9-ICP6gRNA-Neo^{R} | 10076 |

### Example 1

### Design of Guide Sequences of gRNAs

The HSV-1 ICP6 partial sequence (SEQ ID No. 17) containing a GATC insertion was selected as a target and was used to design guide sequences of gRNAs, which are complementary to an ICP6 target sequence of 20 nucleotides covering the GATC insertion.

Among a generated DNA sequence pool, six ICP6 target sequences covering the GATC insertion were picked as candidates. Six ICP6 target sequences and their corresponding PAM sequences were shown in Table 1.

TABLE 1

| | ICP6 target | DNA | PAM |
|---|---|---|---|
| No. | sequence (5′→3′) | strand | sequence |
| 1 | ATCGATCTGCGCCGAGGCGG | − | CGG |
| | (SEQ ID No. 18) | | |
| 2 | GAGATCGATCTGCGCCGAGG | − | CGG |
| | (SEQ ID No. 19) | | |
| 3 | TCCGAGATCGATCTGCGCCG | − | AGG |
| | (SEQ ID No. 20) | | |
| 4 | GCCTCGGCGCAGATCGATCT | + | CGG |
| | (SEQ ID No. 21) | | |
| 5 | ATCGATCTCGGACGTCAGCG | + | AGG |
| | (SEQ ID No. 22) | | |
| 6 | TCGATCTCGGACGTCAGCGA | + | GGG |
| | (SEQ ID No. 23) | | |

(Note:
“+” means sense strand of DNA; “−” means antisense strand of DNA. PAM means protospacer adjacent motif. Underline means the inserted GTAC or its portion.)

Six guide sequences of gRNAs 1-6 each targeting one of ICP6 target sequences 1-6 from Table 1 are shown in Table 2. The gRNAs 1-6 containing these guide sequences were further analyzed to evaluate their cleavage efficiency.

TABLE 2

| | guide sequences | | SEQ |
|---|---|---|---|
| No. | of gRNA 1-6 | Length | ID No. |
| 1 | UAGCUAGACGCGGCUCCGCC | 20 | 24 |
| 2 | CUCUAGCUAGACGCGGCUCC | 20 | 25 |
| 3 | AGGCUCUAGCUAGACGCGGC | 20 | 26 |
| 4 | CGGAGCCGCGUCUAGCUAGA | 20 | 27 |
| 5 | UAGCUAGAGCCUGCAGUCGC | 20 | 28 |
| 6 | AGCUAGAGCCUGCAGUCGCU | 20 | 29 |

### Example 2

### In-Vitro Cleavage Efficiency Assay of Designed gRNAs

A cleavage efficiency assay was performed by using a Cas9 in-vitro cleavage kit (Inovogen Tech. Co., catalog No. PC1400) and following the manufacturer's instructions. In brief, a Cas9 cleavage reaction was conducted by co-incubating a Cas9 protein (from kit), a designed gRNA from Table 2, and an ICP6 target DNA fragment in a Cas9 reaction buffer (from kit) at a temperature of 37° C. for 30 mins, and then the reaction was stopped by incubating at a temperature of 85° C. for 10 mins. The results were observed by DNA gel electrophoresis.

The gRNAs were transcribed and obtained from a synthesized polynucleotide template (as shown below) by using a commercial sgRNA in-vitro transcription kit (Inovogen Tech. Co., catalog No. PC1380) and followed by a purification process.
N²⁰-gttttagagctagaaatagcaagttaaaataaggctagtccgttatcaacttgaaaaagtggcaccgagtcggtgctttt, wherein N²⁰ was a selected ICP6 target sequence shown in Table 1.

A polymerase chain reaction (PCR) was performed to synthesize the ICP6 target DNA fragment (594 bp) by using HSV-1 target strain's genome DNA, as disclosed in Material section as a DNA template, and primer pairs ICP6-F and ICP6-R (SEQ ID No. 30 and 31), wherein the temperatures of denaturation, primer annealing and primer extension were 98° C., 61° C., 72° C., respectively, and reaction cycles were 35.

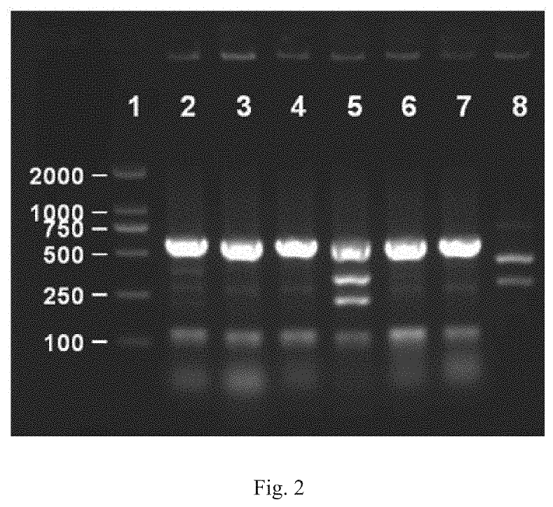

The results of the cleavage efficiency assay are shown in FIG. 2. Gel electrophoresis revealed that gRNA 4 (lane 5) exhibited high cleavage activity, generating DNA cleavage products of two bands which were smaller than 594 bp. The 594 bp band indicated an uncleaved ICP6 target DNA fragment, gRNAs 1, 2, 3, 5 and 6 (i.e. lane 2, 3, 4, 6 and 7), all exhibited weak cleavage activity, generating only a small amount of DNA cleavage products.

According to above results, gRNA 4 has the greatest potential to be an HDR-enhancing gRNA. Therefore. ICP6 target sequence 4 (SEQ ID No. 21) was selected for the following expression plasmid construction and CRISPR/Cas9-mediated homologous recombination.

### Example 3

### Construction of Cas9/gRNA Expression Plasmid pCas9-ICP6gRNA-Neo^{R}

To clone ICP6 target sequence 4 (SEQ ID No. 21) into plasmid pX330-U6-Chimeric_BB-CBh-hSpCas9 (Addgene, catalog No. 42230), two partially complementary oligonucleotides, ICP6gRNA4-F and ICP6gRNA4-R (SEQ ID No. 1 and 2), with 4 nucleotides overhangs were synthesized as below.

(SEQ ID No. 1)
5′-CACC GGCCTCGGCGCAGATCGATCT-3′
(SEQ ID No. 2, reversed)
3′-CCGGAGCCGCGTCTAGCTAGA CAAA-5′

The partially complementary oligos were annealed to form a double-strand DNA with 4 nucleotide overhangs by an annealing program. The double-stranded DNA (containing desired ICP6 target sequence 4) with sticky ends was cloned into a BbsI pre-digested plasmid pX330-U6-Chimeric_BB-CBh-hSpCas9 to obtain a plasmid called pX330-ICP6gRNA. Successful cloning of the ICP6 target sequence 4 was confirmed by PCR with primer pairs U6-seq-F2 and ICP6gRNA4-R (SEQ ID No. 16 and 2) and agarose gel electrophoresis (PCR product is 243 bp).

Then, plasmid pX330-ICP6gRNA was used as a DNA template, and a PCR was performed to amplify U6-ICP6gRNA fragments (SEQ ID No. 5) by using primer pairs MluI-gRNA-F and SpeI-sgRNA-R (SEQ ID No. 3 and 4), wherein temperatures of denaturation, primer annealing and primer extension were 98° C., 58° C., 72° C. respectively, and reaction cycles were 35. The PCR product was analyzed and confirmed by 1% agarose gel electrophoresis (474 bp). The amplified U6-ICP6gRNA fragments were recycled and digested by restriction enzymes MluI and SpeI for further use.

A Cas9 gene was cloned into plasmid pcDNA3.1 (Invitrogen, catalog No. V79020) to obtain a plasmid called pcDNA3.1-Cas9. Then, the obtained plasmid pcDNA3.1-Cas9 was digested by restriction enzyme MluI and SpeI.

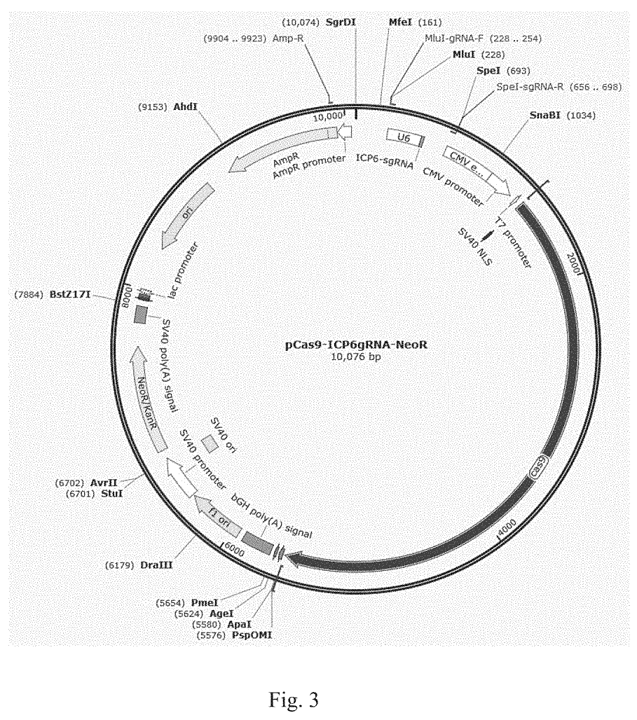

Then, the MluI/SpeI pre-cleaved U6-ICP6gRNA fragments and plasmid pcDNA3.1-Cas9 were ligated via T4 DNA ligase to obtain a plasmid called “pCas9-ICP6gRNA-Neo^{R}” (as shown in FIG. 3 and SEQ ID NO. 36).

Plasmid pCas9-ICP6gRNA-Neo^{R} was then transfected into Transl-T1 Phage Resistant Chemically Competent Cells (TransGen Biotech, catalog No. CD501). Transfected cells were incubated in a selective culture medium at 37° C. over-night. A single clone of transfected cells was picked up, and such transfection was confirmed by PCR with primer pairs MluI-gRNA-F and SpeI-sgRNA-R (SEQ ID No. 3 and 4) and 1% agarose gel electrophoresis (PCR product is 474 bp). The obtained transfected cells containing plasmid pCas9-ICP6gRNA-Neo^{R} were stored for further use.

### Example 4

### Preparation of Vero Cells Transfected with Plasmid pCas9-ICP6gRNA-Neo^{R}

The plasmid pCas9-ICP6gRNA-Neo^{R}-contained transfected cells obtained in example 3 were cultured in LB medium at 37° C. over-night, and then were subjected to a plasmid DNA extraction by using a EasyPure® HiPure Plasmid MaxiPrep Kit (TransGen Biotech, catalog No. EM121) and following the manufacturer's protocol. The extracted plasmid pCas9-ICP6gRNA-Neo^{R} was processed to be linear by use of the restriction enzyme MluI-HF (NEB) in a CutSmart® Buffer (New England Biolabs, catalog No. B7204S) and then was purified.

The obtained linear plasmid pCas9-ICP6gRNA-Neo^{R} was transfected into Vero cells (ATCC® CCL-81™) by using a commercial reagent, Lipofectamine™ 3000 (Invitrogen™, catalog No. L3000008), and following the manufacturer's protocol. Briefly, diluted plasmid DNA was prepared by mixing 5 ug linear plasmid pCas9-ICP6gRNA-Neo^{R}, 5 ul P3000™ reagent (Invitrogen™, catalog No. L3000008) and 250 ul Opti-MEM™ medium (Gibco™, catalog No. 31985088). Diluted Lipofectamine™ 3000 reagent was obtained by mixing 5 ul Lipofectamine™ 3000 reagent and 250 ul Opti-MEM™ medium. A DNA-lipid complex was prepared by mixing above diluted plasmid DNA and diluted Lipofectamine™ 3000 reagent, followed by incubation for 5 mins at room temperature.

For transfection, the DNA-lipid complex (containing linear plasmid pCas9-ICP6gRNA-Neo^{R}) was then co-incubated with Vero cells, which were seeded in a 6-well plate comprising 2 mL complete growth medium at a cell density of 4×10⁵ cells/well. After incubating for 24 hours, 5-10 mL growth medium comprising selective antibiotic G418 (Geneticin™, Gibco™, catalog No. 10131027) with a final concentration of 800 ug/ml was refreshed every two or three days depending on cell growth conditions. Following incubation for about 15 days, the Vero cells transfected with plasmid pCas9-ICP6gRNA-Neo^{R} (hereinafter referred to as “Vero-ICP6 cell”) grew stably and were stored for the following experiment.

### Example 5

### Construction of Homologous Recombination Targeting Vector

To construct a homologous recombination (HR) targeting vector, a targeting polynucleotide (SEQ ID No. 15) comprising upstream/downstream homology arms (SEQ ID No. 12 and 13) and an exogenous EGFP gene (SEQ ID No. 14) were prepared as follows.

The upstream homology arm (UHA; SEQ ID No. 12) was synthesized by PCR, wherein HSV-1 target strain's genome DNA disclosed in the Materials section was used as a DNA template, primer pairs were ICP6-SOE-F1 and ICP6-SOE-R1 (SEQ ID No. 6 and 7), temperatures of denaturation, primer annealing, and primer extension were 98° C., 63° C., 72° C., respectively, and reaction cycles were 35. The PCR product (1534 bp) was confirmed by 1% agarose gel electrophoresis and was then recovered for further use.

The downstream homology arm (DHA; SEQ ID No. 13) was synthesized by PCR, wherein HSV-1 target strain's genome DNA disclosed in Material section was used as a DNA template, primer pairs were ICP6-SOE-F3 and ML40-SOE-R3 (SEQ ID No. 8 and 9), temperatures of denaturation, primer annealing, and primer extension were 98° C., 62° C., 72° C., respectively, and reaction cycles were 35. The PCR product (1194 bp) was confirmed by 1% agarose gel electrophoresis and was then recovered for further use.

The exogenous EGFP gene (SEQ ID No. 14) was synthesized by PCR, wherein pEGFPN1 (Clontech) was used as a DNA template, primer pairs were EGFP-SOE-F2 and EGFP-SOE-R2 (SEQ ID No. 10 and 11), temperatures of denaturation, primer annealing, and primer extension was separately 98° C. 60° C., 72° C. respectively, and reaction cycles were 35. The PCR product (1628 bp) was confirmed by 1% agarose gel electrophoresis and was then recovered for further use.

Then, the upstream homology arm (UHA; SEQ ID No. 12) and the exogenous EGFP gene (SEQ ID No. 14) were joined together by overlap-extension PCR to produce an UHA-EGFP fragment (3133 bp). The UHA-EGFP fragment and downstream homology arm (DHA; SEQ ID No. 13) were joined together by another overlap-extension PCR to produce an UHA-EGFP-DHA fragment (hereinafter referred to as “targeting polynucleotide”; SEQ ID No. 15). The final overlapped PCR product (4300 bp) was confirmed by 1% agarose gel electrophoresis and was then recovered for further use.

The HR targeting vector was obtained by cloning the targeting polynucleotide (SEQ ID No. 15) into a commercial pEASY-Blunt cloning vector (TransGen Biotech, catalog No. CB101-01), which was then transfected into Transl-T1 Phage Resistant Chemically Competent Cells (TransGen Biotech, catalog No. CD501). The transfected cells were incubated in a selective LB medium at 37° C. over-night. A single clone of transfected cells was picked up, and such transfection was confirmed by PCR with primer pairs ICP6-SOE-F1 and ML40-SOE-R3 (SEQ ID No. 6 and 9) followed by 1% agarose gel electrophoresis (PCR product is 4300 bp). The transfected cells thus obtained containing the HR targeting vector were stored for further use.

### Example 6

### CRISPR/Cas9-Mediated Homologous Recombination in HSV-1

The HR targeting vector was harvested from the transfected cells obtained in example 5 by using an EasyPure® HiPure Plasmid MaxiPrep Kit (TransGen Biotech, catalog No. EM121), and followed by being processed to be linear by use of the restriction enzyme EcoRV-HF (New England Biolabs, catalog No. R3195S) in a CutSmart® Buffer (New England Biolabs, catalog No. B7204S). The linear HR targeting vector thus obtained was purified by using a general purification method.

The linear HR targeting vector was transfected into Vero-ICP6 cell obtained in example 4 by using a commercial reagent Lipofectamine™ 3000 (Invitrogen™, catalog No. L3000008) and following the manufacturer's protocol. Briefly, diluted plasmid DNA was prepared by mixing 5 ug linear HR targeting vector, 5 ul P3000™ reagent and 125 ul Opti-MEM™ medium. Diluted Lipofectamine™ 3000 reagent was obtained by mixing 5 ul Lipofectamine™ 3000 reagent and 125 ul Opti-MEM™ medium. A DNA-lipid complex was prepared by mixing above diluted plasmid DNA and diluted Lipofectamine™ 3000 reagent, followed by incubation for 15 mins at room temperature.

Then, for transfection, the DNA-lipid complex (containing linear HR targeting vector) was co-incubated with Vero-ICP6 cells (containing linear plasmid pCas9-ICP6gRNA-Neo^{R}) which were seeded in a 6-well plate comprising 2 mL complete growth medium at a cell density of 4×10⁵ cells/well, at a temperature of 37° C. for 8 hours. After 8 hours incubation, to the above culture medium 2 mL basic culture medium containing HSV-1 target strain disclosed in Material section was added (MOI=1) to cause virus infection. Virus-induced cytopathic effect was observed during culture period. When cytopathic effect increased to 80% (about 48 hours), culture medium containing virus-infected Vero cells was harvested and stored at −80° C. for further use.

### Example 7

### Identification of the Recombinant HSV-1

For identification, the above virus-infected Vero cells harvested in example 6 were subjected to three freeze/thaw cycles (−80° C./30° C.) and then centrifuged at 3000 rpm (ThermoFisher, centrifuge reference no. 75005297; rotor catalog no. 75003331) for 10 mins so as to obtain a supernatant containing recombinant HSV-1. Proteinase K (20 mg/ml) and 10% SDS were added to the supernatant, which was then incubated at 58° C. for 2 hours, 98° C. for 10 mins, and 4° C. for 5 mins. After incubation, centrifugation with 15000 g was performed for 5 mins, and the supernatant was harvested for use as a DNA template in the following PCR experiment.

Two PCRs were used to confirm correct homologous recombination of HSV-1. As shown in FIG. 4, an exogenous gene (EGFP) was inserted by correct homologous recombination, and two primer pairs each was designed for amplifying part of the EGFP gene and a segment of the downstream HR arm, as indicated in FIG. 4.

In first stage PCR, HSV-1 target strain's genome DNA obtained in the Materials section was used as a DNA template, primer pairs were HSV1-GFP-KI-F1 and HSV1-GFP-KI-R1 (SEQ ID No. 32 and 33), temperatures of denaturation, primer annealing, and primer extension were 98° C., 60° C. 72° C., respectively, and reaction cycles were 35. The PCR product (2214 bp) was confirmed by 1% agarose gel electrophoresis and was then recovered for use as a DNA template for next PCR.

In second stage PCR, the obtained first PCR product (2214 bp) was used as a DNA template, primer pairs were HSV1-GFP-KI-F2 and HSV1-GFP-KI-R2 (SEQ ID No. 34 and 35), temperatures of denaturation, primer annealing, and primer extension were 98° C., 60° C., 72° C., respectively, and reaction cycles were 35. The PCR product (2063 bp) was confirmed by 1% agarose gel electrophoresis.

Results for samples 1-4 for the first PCR of recombinant HSV-1 are shown in lanes 2-5, respectively, of FIG. 5 and results of the second stage PCR are shown in lanes 8-11, respectively. A band corresponding to the intended amplicon, beginning in the EGFP gene and extending into the downstream HR arm, were observed for recombinant HSV-1 samples 3 and 4 (lane 4-5 for first stage PCR, and lanes 10-11 for second stage PCR as well), indicating that homologous recombination was completed successfully in those samples.

### Example 8

### Purification of the Recombinant HSV-1

The recombinant HSV-1 was purified by using a plaque purification method. In brief, the Vero cells were seeded in a 6-well plate comprising complete growth medium at a cell density of 8×10⁵ cells/well and cultured at 37° C. over-night. Then, the original medium was removed and 1 mL diluted recombinant virus solution was added to each well to co-incubate at 37° C. for 2 hours. After co-incubation, the remaining virus solution was discarded, and 2 mL complete growth medium containing 1.8% low-melting point agarose (Sigma, catalog No. A9045) was added to form a cover layer. The plates were inverted and incubated at 37° C. The plates were checked daily for cytopathic effect.

After obvious cell lesion was observed (about 48 hours), 2 mL complete growth medium containing 1.8% low-melting point agarose and diluted neutral red (Sigma, catalog No. N2889) was added to form another cover layer. The plates were again inverted and incubated at 37° C. for 24 hours. Green fluorescent plaques were observed by using a fluorescence microscope.

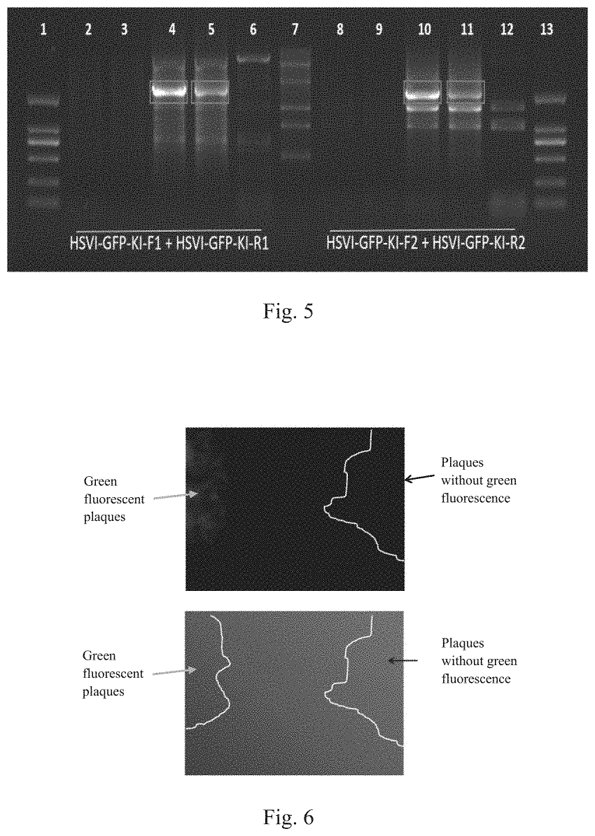

As shown in FIG. 6, the green fluorescent plaques were harvested. Subsequently, the green fluorescent plaques were selected and purified, and plaque purification was performed at least three times until the plaques all became green fluorescent plaques. The green fluorescent plaques indicated that the CRISPR/Cas9-mediated homologous recombination of HSV-1 target strain was successfully completed by use of the disclosed gRNA.

What is claimed is:

1. A guide RNA (gRNA) comprising a guide sequence capable of targeting a Cas9 protein to a target sequence in an ICP6 gene of type 1 herpes simplex virus (HSV-1) to provide a cleavage event, wherein the target sequence comprises a GATC insertion as compared to a wild-type HSV-1.

2. The gRNA according to claim 1, wherein the ICP6 gene expresses an inactivated ICP6 protein caused by inserting a GATC sequence.

3. The gRNA according to claim 1, wherein the ICP6 gene is an inactivated ICP6 gene.

4. The gRNA according to claim 1, wherein the ICP6 gene comprises a sequence corresponding to nucleotides 25-64 of SEQ ID NO. 17.

5. The gRNA according to claim 1, wherein the ICP6 gene comprises SEQ ID NO. 17.

6. The gRNA according to claim 5, wherein the target sequence is a sequence of 20 nucleotides selected from a sequence range corresponding to nucleotides 25-64 of SEQ ID NO. 17.

7. The gRNA according to claim 5, wherein the target sequence comprises a sequence of 20 nucleotides selected from SEQ ID NO. 17.

8. The gRNA according to claim 5, wherein the target sequence comprises a sequence selected from the group consisting of SEQ ID No. 18, 19, 20, 21, 22 and 23.

9. The gRNA according to claim 5, wherein the target sequence is selected from the group consisting of SEQ ID No. 18, 19, 20, 21, 22 and 23.

10. The gRNA according to claim 5, wherein the target sequence is SEQ ID No. 21.

11. The gRNA according to claim 1, wherein the guide sequence comprises a sequence selected from the group consisting of SEQ ID No. 24, 25, 26, 27, 28 and 29.

12. The gRNA according to claim 1, wherein the guide sequence is a sequence selected from the group consisting of SEQ ID No. 24, 25, 26, 27, 28 and 29.

13. The gRNA according to claim 1, wherein the guide sequence is SEQ ID No. 27.

14. A first polynucleotide encoding a gRNA according to claim 1.

15. A vector comprising a first polynucleotide according to claim 14 operably linked to a suitable promoter.

16. The vector according to claim 15, further comprising a second polynucleotide encoding a Cas9 protein.

17. A vector system comprising one vector according to claim 15 and a second vector comprising a second polynucleotide encoding a Cas9 protein.

18. A transformant cell transformed with a vector according to claim 16 or a vector system according to claim 17, which is capable of expressing a gRNA and a Cas9 protein.

19. The transformant cell according to claim 18, which is a Vero cell.

20. A Cas9/gRNA complex comprising a gRNA according to claim 1 and a Cas9 protein.

21. A gene-editing system for HSV-1, comprising:
(a) a HSV-1 strain comprising a target sequence in an ICP6 gene, wherein the target sequence comprises a GATC insertion as compared to a wild-type HSV-1;
(b) a vector according to claim 16, which is capable of expressing a gRNA and a Cas9 protein; and
(c) a targeting polynucleotide comprising an upstream homology arm, an exogenous gene and a downstream homology arm sequentially, wherein the upstream homology arm and the downstream homology arm are separately homologous to a 5′ region and a 3′ region of a target domain of said HSV-1, wherein the target sequence is located within the target domain of said HSV-1.

22. A gene-editing method for generating a recombinant HSV-1, comprising steps of:
(a) providing a HSV-1 strain, wherein the HSV-1 strain comprises a target sequence in an ICP6 gene, and the target sequence comprises a GATC insertion as compared to a wild-type HSV-1;
(b) constructing a vector according to claim 16, which is capable of expressing a gRNA and a Cas9 protein;
(c) preparing a linear DNA including a targeting polynucleotide, which is capable of inserting an exogenous gene into a target domain of said HSV-1 via homologous recombination;
(d) transforming the vector or the vector system into a transformant cell to obtain a first transformant;
(e) transforming the linear DNA into the first transformant to obtain a second transformant; and
(f) infecting the second transformant with the HSV-1 strain to cause CRISPR/Cas9-mediated homologous recombination to occur in the second transformant, wherein the gRNA targets the Cas9 protein to the target sequence to provide a specific cleavage event, and then the exogenous gene is inserted into the target domain via homologous recombination.

23. The gene-editing method according to claim 22, wherein the ICP6 gene comprises SEQ ID NO. 17.

24. The gene-editing method according to claim 22, wherein the target sequence comprises a sequence selected from the group consisting of SEQ ID No. 18, 19, 20, 21, 22 and 23.

25. The gene-editing method according to claim 22, wherein the target sequence is selected from the group consisting of SEQ ID No. 18, 19, 20, 21, 22 and 23.

26. The gene-editing method according to claim 22, wherein the target sequence is SEQ ID No. 21.

27. The gene-editing method according to claim 22, wherein the targeting polynucleotide in step (c) comprises an upstream homology arm, the exogenous gene and a downstream homology arm sequentially; wherein the upstream homology arm and the downstream homology arm are separately homologous to a 5′ region and a 3′ region of a target domain of said HSV-1, wherein the target sequence is located within the target domain of said HSV-1.

28. The gene-editing method according to claim 27, wherein the upstream homology arm comprises SEQ ID No. 12.

29. The gene-editing method according to claim 27, wherein the downstream homology arm comprises SEQ ID No. 13.

30. A recombinant HSV-1 generated by the gene-editing method according to claim 22.
